(19) United States
(12) Patent Application Publication
(10) Pub. No.: US 2026/0081012 A1
(43) Pub. Date: Mar. 19, 2026

(54) MEDICAL IMAGING APPARATUS

(71) Applicant: SHIMADZU CORPORATION, Kyoto-shi (JP)

(72) Inventor: Masahiro TANAKA, Kyoto-shi (JP)

(21) Appl. No.: 19/133,625

(22) PCT Filed: Sep. 11, 2023
(86) PCT No.: PCT/JP2023/033018

§ 371 Date: May 28, 2025

(30) Foreign Application Priority Data
Nov. 29, 2022 (JP) 2022-190184

(51) Int. Cl. G16H 40/63; A61B 6/46

(52) U.S. Cl. CPC G16H 40/63; A61B 6/461; A61B 6/467

(57) ABSTRACT

Provided is a medical imaging apparatus capable of flexibly executing appropriate support for an operator who performs complicated imaging. The X-ray imaging apparatus of the present invention includes a console that causes an operator's description to be input, an information processing unit that associates an imaging type with an operator's description content, a memory that stores the operator's description content in association with the imaging type, an imaging type acquisition unit that acquires an imaging type to be executed, and a display unit that reads the description content of the operator corresponding to the acquired imaging type from the memory and displays the description content.

### TECHNICAL FIELD

The present invention relates to a medical imaging apparatus such as an X-ray imaging apparatus including a monitor.

### BACKGROUND ART

Patent Literature 1 describes a configuration of a medical imaging apparatus in which an operator wears a kneeboard and operates the apparatus while confirming the matters described in the kneeboard. The kneeboard is a memo in which instruments necessary for execution of the medical stage, cautions in implementation, and the like are written. If the kneeboard is created before surgery, this memo can be referred to during surgery and can be used as supplementary information for safely performing surgery. In addition, Patent Literature 1 describes determination of bleeding according to the use time of gauze. A clock may be used for the determination.

### CITATION LIST

### Patent Literature

Patent Literature 1: JP 2008-86756 A

### SUMMARY OF INVENTION

### Technical Problem

However, the medical imaging apparatus according to the above-described configuration has not been sufficiently pursued regarding support for the operator. In a case of performing complicated imaging in which the operator has to perform a plurality of procedures, in order to smoothly perform the imaging, a function of supporting the operator who pays attention is necessary as a caution when performing the imaging. Since the procedure varies depending on the imaging type, the caution required of the operator for each procedure varies depending on the imaging type. For example, even in a medical imaging apparatus in which the imaging type is fixed to one type, the caution required of the operator may change as the imaging proceeds. In order to reliably support such an operator, flexibility is required for the reference content.

Conventional apparatuses are not configured by sufficiently considering such circumstances. As a result, the operator has to rely on a tool outside the apparatus as in Patent Literature 1. Such a configuration cannot be said to be optimal in terms of hygiene, and a configuration that can complete support for an operator with a medical imaging apparatus is desired.

The present invention has been made in view of such circumstances, and an object of the present invention is to provide a medical imaging apparatus capable of flexibly executing appropriate support for an operator who performs complicated imaging.

### Solution to Problem

In order to achieve such an object, a first aspect of the present invention has the following configuration.

A medical imaging apparatus including: a description input unit that inputs a description of an operator; a description content processing unit that associates an imaging type with the description content of the operator; a memory that stores the description content of the operator in association with the imaging type; an imaging type acquisition unit that acquires an imaging type to be executed; and a display unit that displays the description content of the operator corresponding to the acquired imaging type.

According to the first aspect described above, a description of an operator is input, and the description is associated with an imaging type. Since the operator can freely describe, the information related to the imaging type can be held in the memory according to the convenience of the operator. Then, in a case where imaging for a certain imaging type is executed, the display unit displays the description content of the operator corresponding to the imaging type to be executed. Since the operator can perform imaging while referring to the display content, smooth and reliable imaging can be performed with appropriate support. Moreover, since the support regarding the photographing does not depend on a tool outside the apparatus, the imaging is optimized in terms of hygiene.

In order to achieve the above-described object, a second aspect of the present invention may have the following configuration.

A medical imaging apparatus including: a setting value input unit that inputs a setting value of time by an operator; a setting value processing unit that associates an imaging type with the setting value; a memory that stores the setting value in association with the imaging type; an imaging type acquisition unit that acquires an imaging type to be executed; and a display unit that displays a timer capable of measuring time indicated by a setting value corresponding to the acquired imaging type.

According to the second aspect described above, the setting value of the time by the operator is input, and the setting value is associated with the imaging type. Since the operator can freely input the setting value, the timer measurement time according to the imaging type can be held in the memory according to the convenience of the operator. Then, in a case of executing imaging for a certain imaging type, the display unit displays a timer capable of measuring time indicated by a setting value corresponding to the imaging type to be executed. Since the operator can perform imaging while referring to the timer, smooth and reliable imaging can be performed with appropriate support. Moreover, since the support regarding the photographing does not depend on a tool outside the apparatus, the imaging is optimized in terms of hygiene.

### Advantageous Effects of Invention

According to the medical imaging apparatus according to the present invention, assistance to an operator can also be completed by an apparatus configuration. That is, according to the present invention, it is possible to flexibly support the operator for each imaging type. This is because the operator can appropriately set the reference content in advance according to his/her convenience through the input unit. The present invention assists an operator by acquiring an imaging type to be executed and performing display matching the imaging type. Therefore, it can be said that the medical imaging apparatus according to the present invention is optimal in terms of hygiene without requiring the operator to rely on a tool outside the apparatus.

### BRIEF DESCRIPTION OF DRAWINGS

FIG. 1 is a functional block diagram for explaining an overall configuration of an X-ray imaging apparatus according to an embodiment.

FIG. 2 is a layout diagram of each unit constituting a console according to an embodiment.

FIG. 3 is a schematic diagram for explaining stored contents of a memory according to an embodiment.

FIG. 4 is a schematic diagram for explaining stored contents of a memory according to an embodiment.

FIG. 5 is a schematic diagram for explaining stored contents of a memory according to an embodiment.

FIG. 6 is a flowchart for explaining pre-imaging setting processing according to the embodiment.

FIG. 7 is a schematic diagram for explaining display contents of a display unit in pre-imaging setting processing.

FIG. 8 is a schematic diagram for explaining a state in which an operator makes a description in pre-imaging setting processing.

FIG. 9 is a flowchart for explaining description content display processing according to the embodiment.

FIG. 10 is a schematic diagram for explaining display content of a display unit in description content display processing.

FIG. 11 is a schematic diagram for explaining display content of a display unit in description content display processing.

FIG. 12 is a schematic diagram for explaining display content of a display unit in description content display processing.

FIG. 13 is a schematic diagram for explaining display content of a display unit in description content display processing.

FIG. 14 is a schematic diagram for explaining display content of a display unit in description content display processing.

FIG. 15 is a schematic diagram for explaining display content of a display unit in description content display processing.

### DESCRIPTION OF EMBODIMENTS

Hereinafter, a medical imaging apparatus according to an embodiment will be described with reference to the drawings. Hereinafter, drip infusion pyelography (DIP) imaging related to a urinary organ using an X-ray imaging apparatus will be described as an example. The present invention relates to a general medical imaging apparatus including a monitor, and an X-ray imaging apparatus is an example of a medical imaging apparatus applicable to the present invention.

### EXAMPLES

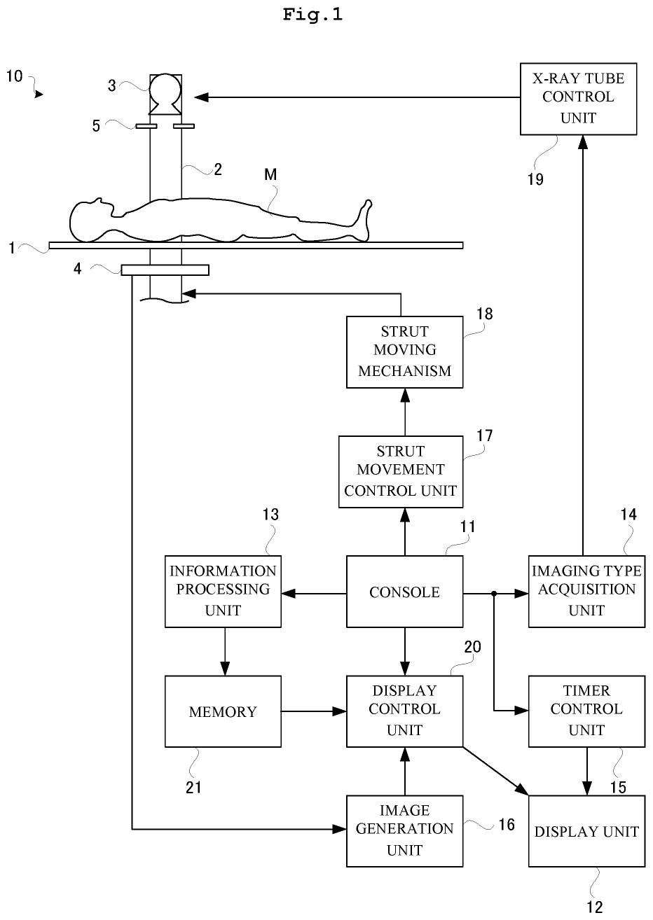

As illustrated in FIG. 1, an X-ray imaging apparatus 10 according to an embodiment includes a top plate 1 on which a subject M in a horizontal posture is placed, a strut 2 that is movable along a body axis of the subject M and extends in a vertical direction, an X-ray tube 3 that is disposed at a distal end portion of the strut 2 and is located above the top plate 1 and irradiates the subject M with an X-ray, a detector 4 that is located below the top plate 1 and detects the X-ray that has passed through the subject M, and a collimator 5 that is provided at a position sandwiched between the top plate 1 and the X-ray tube 3 and restricts an irradiation position of the X-ray beam with respect to the subject M.

A console 11 is provided for the operator to give various instructions to the X-ray imaging apparatus 10. Specific examples of the instruction given by the operator include an instruction to start X-ray irradiation. In addition, the console 11 is configured to receive the operator's character input, which will be described later. The console 11 of the embodiment corresponds to a description input unit and a setting value input unit of the present invention.

A display unit 12 is, for example, a liquid crystal monitor, and can display an electronic medical record of a patient, a live image which is a moving image, and a still image (X-ray image) for diagnosis. In addition, the display unit 12 is configured to perform display related to the operator's character input and display to support the X-ray imaging operation of the operator, which will be described later.

An information processing unit 13 is configured to transmit the description content input by the operator through the console 11 to a memory 21 in association with the imaging type. A specific configuration of the information processing unit 13 will be described later. The information processing unit 13 according to the embodiment corresponds to a description content processing unit and a setting value processing unit according to the present invention.

An imaging type acquisition unit 14 is configured to acquire an imaging type related to control of the X-ray tube 3 such as a tube current, a tube voltage, and a pulse width from a radiology information system (RIS). The imaging type acquisition unit 14 transmits the acquired imaging type to an X-ray tube control unit 19 that controls the X-ray tube 3. The X-ray tube control unit 19 controls the X-ray tube 3 based on the transmitted imaging type. The imaging type acquisition unit 14 also transmits the acquired imaging type to the detector 4. The detector 4 operates based on the transmitted imaging type. In addition to the RIS, the imaging type acquisition unit 14 of the embodiment can also acquire the imaging type by an input through the console 11 of the operator. As described above, the X-ray imaging apparatus 10 can change the imaging type by the setting of the operator without depending on the RIS.

A timer control unit 15 controls a timer icon displayed on the display unit 12. The timer icon is configured to display the remaining time on the display unit 12, and operates under the control of the timer control unit 15. The timer control unit 15 starts countdown using the timer icon in accordance with an instruction from the operator through the console 11. A specific configuration of the timer icon will be described later. A timer icon 51 of the embodiment corresponds to the timer of the present invention.

An image generation unit 16 acquires the X-ray detection signal output from the detector 4 and generates an X-ray image. Examples of the generated X-ray image include frames constituting a live image for alignment of a subject and an X-ray image for diagnosis. The image generation unit 16 transmits the generated X-ray image to the display unit 12, and the display unit 12 displays the transmitted X-ray image.

A strut movement control unit 17 controls the movement of the strut 2 in the longitudinal direction of the top plate 1. The strut movement control unit 17 recognizes the moving distance and the moving direction of the strut 2 based on the input through the console 11 of the operator. Then, the strut movement control unit 17 controls a strut moving mechanism 18 based on the recognized moving distance and moving direction of the strut 2. The strut moving mechanism 18 is a mechanism for supporting the strut 2, and moves the strut 2 to a desired position of the operator by moving the strut 2.

Based on an instruction from the imaging type acquisition unit 14, the X-ray tube control unit 19 supplies a predetermined tube voltage and a predetermined tube current to the X-ray tube 3, and causes the X-ray tube 3 to output an X-ray suitable for imaging.

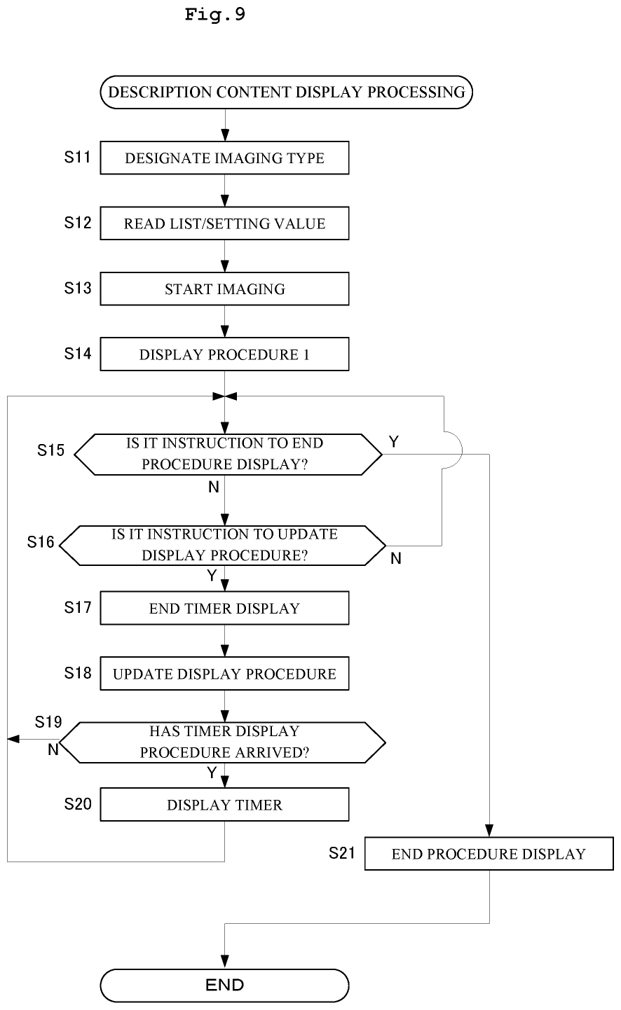

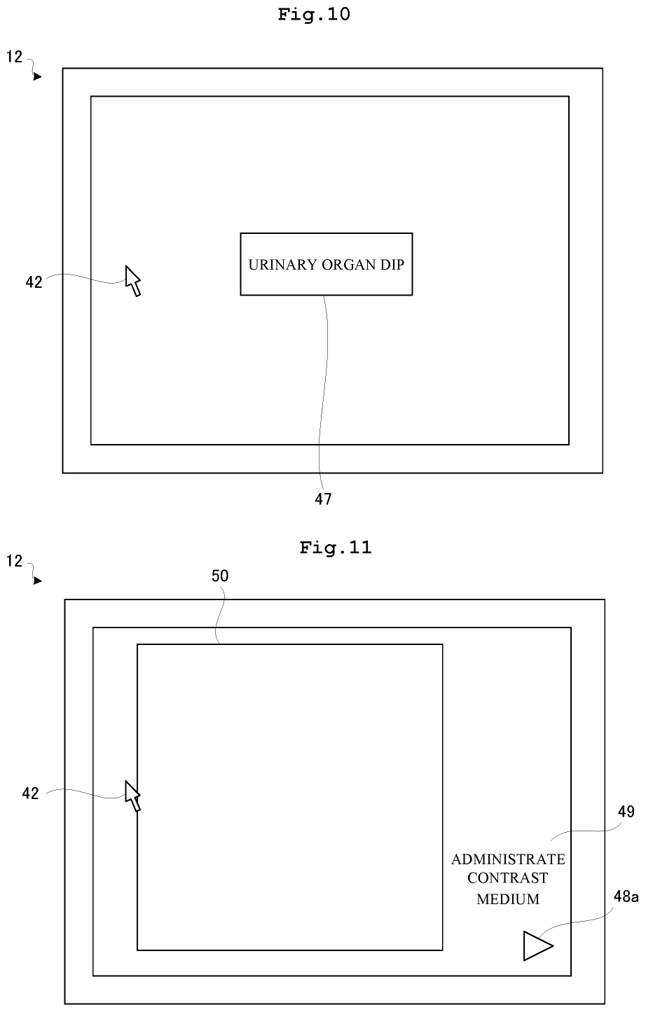

A display control unit 20 is configured to determine the display content of the display unit 12. The display contents in FIGS. 10 to 15 and the flowchart in FIG. 9 to be described later are realized by the operation of the display control unit 20.

The memory 21 stores information necessary for the operation of the X-ray imaging apparatus 10, such as programs, various parameters, and data on the patient necessary for the operations of the information processing unit 13, the imaging type acquisition unit 14, the timer control unit 15, the image generation unit 16, the strut movement control unit 17, the X-ray tube control unit 19, and the display control unit 20. The memory 21 stores the description content input by the operator through the console 11 in association with the imaging type, and this configuration will be described later.

The information processing unit 13, the imaging type acquisition unit 14, the timer control unit 15, the image generation unit 16, the strut movement control unit 17, the X-ray tube control unit 19, and the display control unit 20 of the embodiment are all implemented by a central processing unit (CPU) mounted on the X-ray imaging apparatus 10 executing various programs. The X-ray imaging apparatus 10 of the embodiment is not limited to the configuration in which a single CPU realizes each unit, and a plurality of CPUs can be replaced with the configuration. The same applies to the above-described memory 21. That is, the X-ray imaging apparatus 10 of the embodiment is not limited to the configuration in which the single memory 21 realizes each unit, and a plurality of memories can be replaced with the single memory.

In addition, the X-ray imaging apparatus 10 of the embodiment includes a collimator driving mechanism for adjusting the opening degree of the collimator 5 and a collimator control unit, but these configurations are not illustrated in FIG. 1.

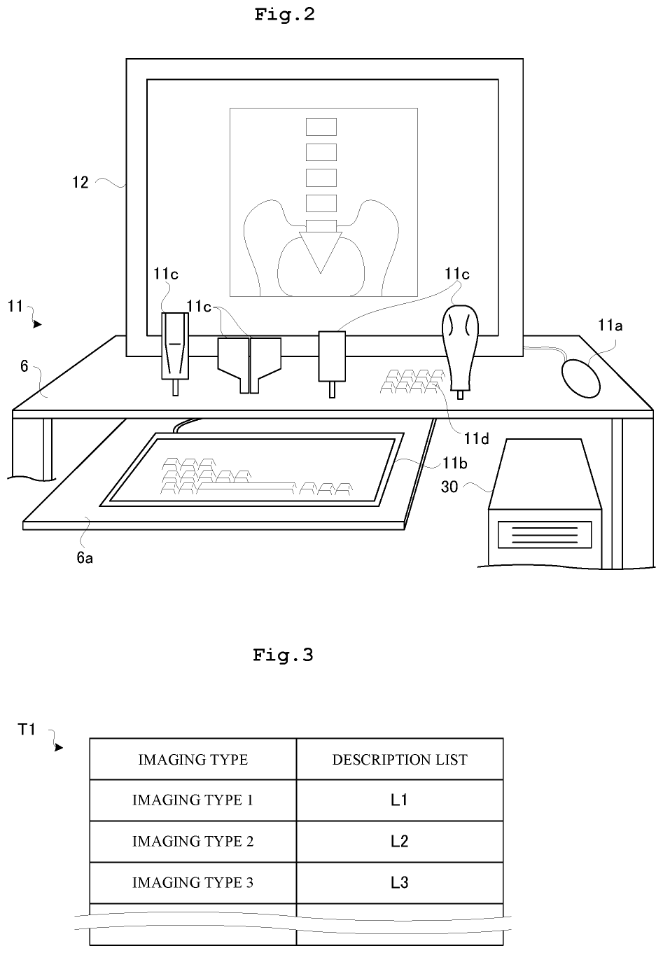

FIG. 2 specifically illustrates the console 11 and the display unit 12 of the embodiment. The console 11 of the embodiment includes various human interfaces provided on an operation desk 6 to which the operator heads. That is, the console 11 includes a mouse 11a that moves a pointer on the display unit 12, a keyboard 11b that inputs characters to the display unit 12, various joysticks 11c that the operator controls the drive unit of the X-ray imaging apparatus 10 such as the collimator 5 and the strut 2, and a control key 11d that inputs an instruction regarding imaging such as an instruction of X-ray irradiation by the operator and forced termination of imaging. Since the keyboard 11b may be unnecessary in many cases and a large size, it is placed on a drawer 6a that can be drawn out from the back side of the main plate of the operation desk 6. Other components of the console 11 are provided on the main plate of the operation desk 6. The main plate is a load-bearing top plate on which a monitor constituting the display unit 12 is placed.

A tower-type computer 30 is disposed below the main plate of the operation desk 6. The computer 30 is an information processing apparatus for realizing the information processing unit 13, the imaging type acquisition unit 14, the timer control unit 15, and the display control unit 20 in the X-ray imaging apparatus 10. The display unit 12, the mouse 11a, and the keyboard 11b are connected to the computer 30.

Details of the information stored in the memory 21 will be described below. FIG. 3 illustrates a configuration of a description list relation table T1 stored in the memory 21. In the description list relation table T1, an imaging type and a description list are associated, a description list L1 is associated with an imaging type 1, a description list L2 is associated with an imaging type 2, and a description list L3 is associated with an imaging type 3. The description list L1, the description list L2, and the description list L3 are tables stored in the memory 21. A specific example of the description list will be described later. The imaging type 1 is an imaging type corresponding to DIP imaging, and when DIP imaging is performed, the description list L1 corresponding to the imaging type 1 is read from the memory 21 and displayed on the display unit 12.

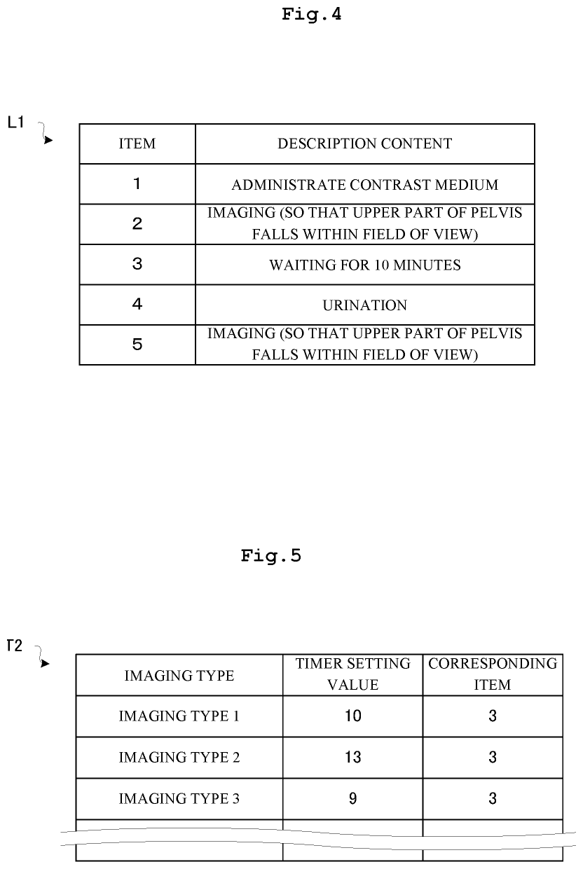

FIG. 4 illustrates content of the description list L1 related to DIP imaging of the imaging type 1. The description list L1 includes a plurality of items, and the items are associated with description contents. In a first item of the description list L1, a character string “administrate contrast medium” is stored. Similarly, a character string “imaging (so that the upper part of the pelvis fits in the field of view)” is stored in the second item, and a character string “waiting for 10 minutes” is stored in the third item. In the fourth item, a character string “urination” is stored, and in the fifth item, a character string “imaging (so that the upper part of the pelvis falls within the field of view)” is stored. As described above, the description list of the embodiment is a table in which the character string and the number of items are associated with each other.

The operator can freely describe and store the character strings constituting the description list in the memory 21 before imaging. The description list indicates the order in which the character strings are displayed on the display unit 12, and according to the description list L1, the characters of “administrate contrast medium”, “imaging (so that the upper part of the pelvis falls within the field of view)”, “waiting for 10 minutes”, “urination”, and “imaging (so that the upper part of the pelvis falls within the field of view)” are displayed on the display unit 12 in this order. Note that the descriptions such as “administrate contrast medium”, “imaging”, “waiting for 10 minutes”, and “urination” are imaging procedures referred to by the operator at the time of imaging. The description “so that the upper part of the pelvis falls within the field of view”is a check content that should be noted by the operator at the time of imaging.

The X-ray imaging apparatus 10 of the embodiment can create a different imaging type for each of a plurality of operators. Therefore, even if the same DIP imaging is performed as the imaging, the description list L1 can be a description suitable for a certain operator, and the description list L2 can be a description suitable for another operator.

FIG. 5 illustrates a configuration of a timer setting value association table T2 stored in the memory 21. In the timer setting value association table T2, an imaging type and a timer setting value are associated, a timer setting value “10” is associated with the imaging type 1, a timer setting value “13” is associated with the imaging type 2, and a timer setting value association table T2 “9” is associated with the imaging type 3. The imaging type 1 is an imaging type corresponding to the DIP imaging, and when the DIP imaging is executed, the timer setting value “10” corresponding to the imaging type 1 is read from the memory 21 and displayed on the display unit 12. The timer setting value designates an initial value indicated by a timer icon displayed on the display unit 12. Assuming that the timer setting value related to the imaging type 1 is “10”, the initial value of the timer icon displayed on the display unit 12 during imaging related to the imaging type 1 is 10 minutes. The operator can accurately know the course of 10 minutes during imaging by using this timer icon.

Further, as can be seen with reference to FIG. 5, in the timer setting value association table T2, not only the timer setting value but also the corresponding item is related to the imaging type. The corresponding item designates one of the items constituting the description list. For example, according to the timer setting value association table T2, the corresponding item related to the imaging type 1 is “3”. The numerical value of the corresponding item indicates the timing at which the timer icon is displayed. Assuming that the corresponding item related to the imaging type 1 is “3”, the timer icon is displayed on the display unit 12 at the timing when the third item “waiting for 10 minutes” of the description list L1 is displayed on the display unit 12.

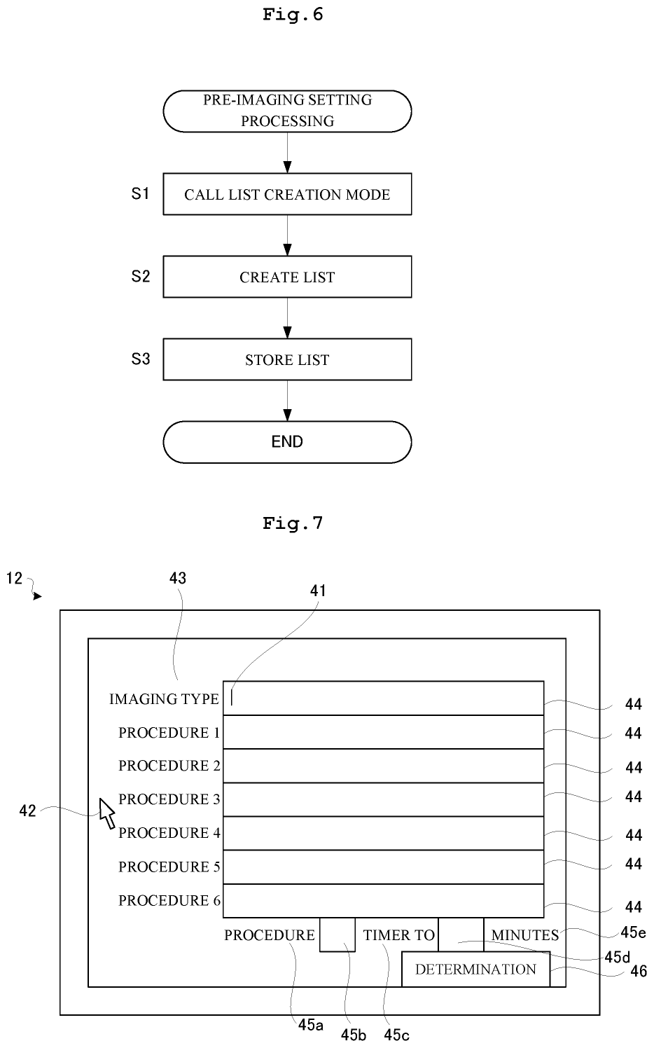

The operator can freely set the character string, the timer setting value, and the numerical value of the corresponding item constituting the description list through the pre-imaging setting processing. The pre-imaging setting processing is processing in which the operator creates each table using the mouse 11a and the keyboard 11b before imaging. Hereinafter, details of the pre-imaging setting processing will be described with reference to FIG. 6 and the like.

### Pre-Imaging Setting Processing

Step S1: When the operator instructs the X-ray imaging apparatus 10 to call a list creation mode prepared in the X-ray imaging apparatus 10 through the console 11, the display control unit 20 causes the display unit 12 to display a list creation screen including a plurality of input boxes as illustrated in FIG. 7. Details of the list creation screen will be described. In the list creation screen, a column 43 having items of “imaging type”, “Procedure 1”, “Procedure 2”, “Procedure 3”, “Procedure 4”, “Procedure 5”, and “Procedure 6” is displayed in the left part of the display unit 12, and an input box 44 corresponding to each item constituting the column 43 is displayed in the central part of the display unit 12. In each input box 44, the operator can freely describe characters. Regarding the term of the imaging type, the predetermined imaging type stored in the memory 21 of the X-ray imaging apparatus 10 may be selected by the operator using a pull-down menu. When the imaging type selected by the operator is matched with the imaging type input to the X-ray imaging apparatus 10 by the RIS, the X-ray imaging apparatus 10 can read the description list corresponding to the imaging type designated by the RIS at the time of imaging and execute imaging.

As illustrated in FIG. 7, on the list creation screen, a character string 45a “procedure”, a numerical value input box 45b, a character string 45c “timer to”, a setting value input box 45d, and a character string 45e “minutes” are displayed in this order at the bottom of the screen. These displays allow the operator to input designation regarding the timer icon. In the numerical value input box 45b, a procedure for displaying the timer icon can be designated, and in the setting value input box 45d, time to be measured with the timer icon can be set.

In the list creation mode, the X-ray imaging apparatus 10 is in a state of receiving an operator's input through the mouse 11a and the keyboard 10b. In FIG. 7, a cursor 41 related to character input by the keyboard 11b is displayed in the input box 44 associated with the term of the imaging type, and a pointer 42 movable by the mouse 11a is displayed on the display unit 12 separately from the cursor 41.

A determination icon 46 is displayed at the bottom of the list creation screen, and when the operator operates the determination icon 46 through the mouse 11a, the description content is determined and a description list is created.

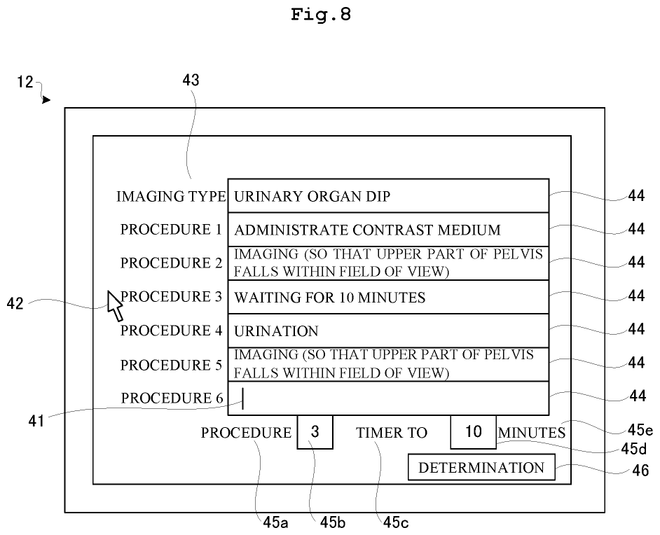

Step S2: FIG. 8 illustrates a state when the operator finishes creating the description list regarding the DIP imaging on the list creation screen. The operator at this time can input characters “urinary organ DIP” in the input box 44 of the imaging type, input characters “administrate contrast medium” in the input box 44 of Procedure 1, input characters “imaging (so that the upper part of the pelvis falls within the field of view)” in the input box 44 of Procedure 2, input characters “waiting for 10 minutes” in the input box 44 of Procedure 3, input characters “urination” in the input box 44 of Procedure 4, and input characters “imaging (so that the upper part of the pelvis falls within the field of view)” in the input box 44 of Procedure 5. In addition, the operator can input a numerical value “3” in the numerical value input box 45b and a setting value “10” in the setting value input box 45d. When the operator operates the determination icon 46 through the mouse 11a in the state of FIG. 8, the description content is determined, and the description list L1 described in FIG. 4 is created. The description list L1 is a list of character strings input to the input box 44 related to the procedure.

The relevance between the description list L1 and the imaging type is held in the description list relation table T1 (see FIG. 3) created by the information processing unit 13. The relevance between the numerical value and the setting value related to the timer display and the imaging type is held in the timer setting value association table T2 (see FIG. 5) created by the information processing unit 13.

Furthermore, in a case where different imaging types are prepared for each operator for the same DIP imaging, a plurality of similar lists having similar procedures is created. At this time, for example, it is possible to input a waiting time different for each operator for Procedure 3 having a waiting time, and it is also possible to set the description to contents or expressions suitable for the operator for other procedures.

As described above, the X-ray imaging apparatus 10 of the embodiment is configured to be able to flexibly cope with different operators.

Step S3: Each table created in the above-described steps is stored in the memory 21. Each table stored in the memory 21 is referred to in description content display processing executed when the operator actually performs imaging.

Hereinafter, the description content display processing will be described in detail with reference to FIG. 9 and the like. <Description Content Display Processing>

Step S11: First, the display control unit 20 recognizes that imaging performed by this is the imaging type 1 related to DIP imaging. This recognition is performed by acquiring information regarding imaging from the RIS or by designating the operator through the console 11. In a case where different imaging types are set by the operator, the imaging type is directly designated by the operator through the console 11. When the recognition of the imaging type is completed, a character string 47 of “urinary organ DIP” meaning the imaging type 1 received from the RIS is displayed on the display unit 12 as illustrated in FIG. 10. The characters of the “urinary organ DIP” may be held in the memory 21 by overwriting the “imaging type 1” of the description list relation table T1 described with reference to FIG. 3, or a table associating the imaging type and the imaging name “urinary organ DIP” may be stored in the memory 21 separately from the tables according to FIGS. 3, 4, and 5, and the character string 47 may be displayed on the basis of the table. In a case where a plurality of inspection types are prepared for each operator, the character strings 47 such as “urinary organ DIP (A)” and “urinary organ DIP (B)” are displayed so that the imaging types can be distinguished. Therefore, in this case, the imaging names stored in the memory 21 are “urinary organ DIP (A)” for the imaging type 1, “urinary organ DIP (B)” for the imaging type 2, and the like.

Step S12: Subsequently, the tables according to FIGS. 3, 4, and 5 stored in the memory 21 are referred to, and the description list “description list L1”, the numerical value “3”, and the setting value “10” corresponding to the urinary organ DIP (imaging type 1) are read from the memory 21.

Step S13: The operator manipulates the console 11 to instruct initiation of the urinary organ DIP.

Step S14: When the image capturing is started, as illustrated in FIG. 11, the display unit 12 displays “administrate contrast medium”, which is the first item of the description list L1, at the end of the screen as a comment 49. In this manner, the character string described by the operator for Procedure 1 is displayed on the display unit 12. An image display area 50 in which an image is displayed is provided in the central portion of the display unit 12, and the comment 49 is displayed on the display unit 12 while avoiding the image display area 50.

A forward icon 48a for sending an item is displayed around the comment 49 on the display unit 12 while avoiding the image display area 50. The operator can operate the forward icon 48a through the mouse 11a. Thereafter, the process proceeds to step S15.

Step S15: In this step, it is determined whether the operator has instructed to end the procedure display. If the determination is false, the process proceeds to step S16, and if true, the process proceeds to step S21. The following description will be made assuming that the operator does not designate the end of the procedure display at this time point.

Step S16: Subsequently, it is determined whether there is an instruction to update the display procedure by the operator. The determination is true in a case where the operator operates the forward icon 48a on the display unit 12 through the mouse 11a. In a case where the forward icon 48a is not operated and the determination is false, the process returns to step S15. Therefore, the processing repeats steps S15 and S16, and a standby state is established until there is an instruction to end the procedure display or the forward icon 48a is operated.

Step S17: When the operator operates the forward icon 48a on the display unit 12 through the mouse 11a and step S16 is true, the process proceeds to step S17, and the display of the timer icon on the display unit 12 ends. When the step is executed for the first time, nothing happens in the step since the timer icon is not displayed, and the process proceeds to the next step.

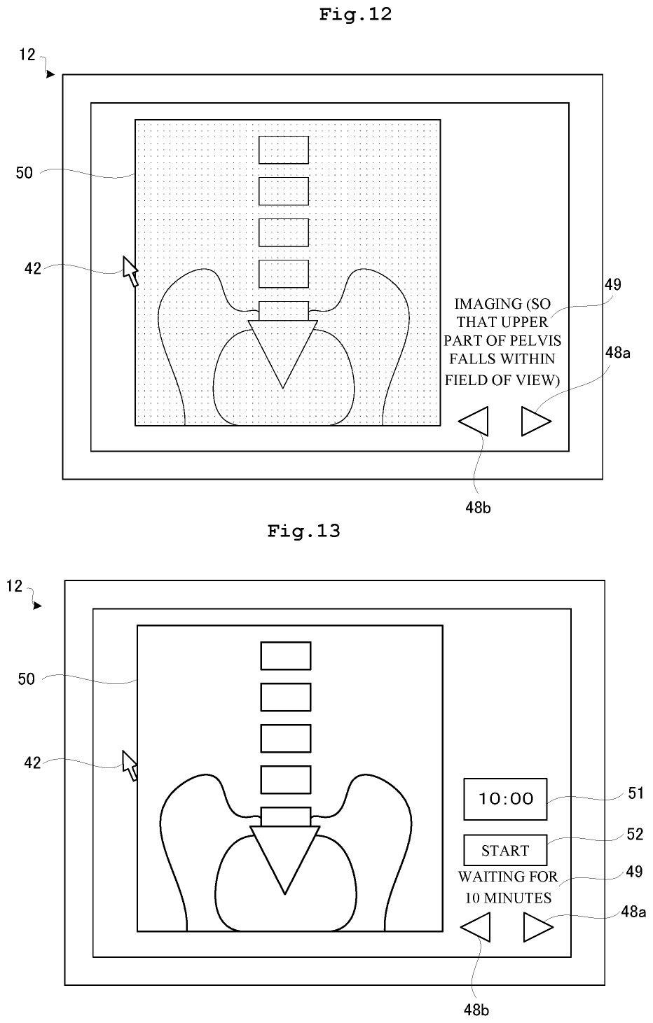

Step S18: Thereafter, as illustrated in FIG. 12, the display unit 12 displays “imaging (so that the upper part of the pelvis fits in the field of view)”, which is the second item of the description list L1, as a comment 49 at the end of the screen. In this manner, the character string described by the operator for Procedure 2 is displayed on the display unit 12, and the displayed procedure is updated. That is, the display of the comment 49 is updated from Procedure 1 to Procedure 2.

Step S19: Thereafter, it is determined whether the displayed procedure is a timer display procedure. The determination is true in a case where the numerical value indicating the order number of the procedure displayed as the comment 49 in the list matches the numerical value stored in the timer setting value association table T2 illustrated in FIG. 5. Since the numerical value corresponding to the imaging type 1 in the timer setting value association table T2 is 3 and the second procedure is displayed in FIG. 12, the determination is false and the process returns to step S15.

FIG. 12 illustrates display contents of the display unit 12 in steps S17 to S19. The operator at this time is in the middle of capturing a live image with the support of the comment 49 so that the upper part of the pelvis falls within the field of view, and a fluoroscopic image (live image) of the subject is displayed in the image display area 50. At this time, around the comment 49 on the display unit 12, in addition to the forward icon 48a for sending the item, a backward icon 48b for returning the item is displayed while avoiding the image display area 50. Similarly to the forward icon 48a, the operator can operate the backward icon 48b through the mouse 11a.

As described above, the processing returns to step S15 when step S19 ends, but at this time, the operator is in the middle of imaging and does not instruct to end the procedure display, and thus the process proceeds to step S16. Then, steps S15 and S16 are repeated until the operator operates the forward icon 48a or the backward icon 48b through the mouse 11a. When the operator operates the forward icon 48a through the mouse 11a, the process proceeds to step S17, but at this time point, since the timer icon is not displayed on the display unit 12, nothing happens. Then, the process proceeds to step S18, and as illustrated in FIG. 13, the display unit 12 displays “waiting for 10 minutes”, which is the third item of the description list L1, at the end of the screen as a comment 49. In this manner, the character string described by the operator for Procedure 3 is displayed on the display unit 12, and the displayed procedure is updated. That is, the display of the comment 49 is updated from Procedure 2 to Procedure 3.

On the other hand, when the operator operates the backward icon 48b through the mouse 11a, the process proceeds to step S17, but at this time point, since the timer icon is not displayed on the display unit 12, nothing happens. Then, the process proceeds to step S18, and the display unit 12 displays “administrate contrast medium”, which is the first item in the description list L1, as the comment 49 at the end of the screen. In this manner, the character string described by the operator for Procedure 1 is displayed on the display unit 12, and the displayed procedure is updated. That is, the display of the comment 49 is updated from Procedure 2 to Procedure 1.

As described above, according to the X-ray imaging apparatus of the embodiment, the comment 49 is reversely sent, and the procedure can easily be re-executed. In addition, it is also possible to refer to the procedure related to the execution again by sending the comment 49 forward and confirming the procedure before actually executing the procedure and then sending the comment 49 backward.

It is assumed that the operator operates the forward icon 48a through the mouse 11a and “waiting for 10 minutes” which is the third item of the description list L1 is displayed as the comment 49 on the display unit 12. Thereafter, the process proceeds to step S19. In this step, it is determined whether the displayed procedure is a timer display procedure. Since the numerical value corresponding to the imaging type 1 in the timer setting value association table T2 is 3 and the third procedure is displayed in FIG. 13, the determination is true, and the process proceeds to step S20.

Step S20: Around the comment 49 on the display unit 12, in addition to the forward icon 48a and the backward icon 48b, the timer icon 51 with characters meaning 10 minutes indicated by the stored setting value such as “10:00” and a start icon 52 describing “start” related to the timer operation are displayed while avoiding the image display area 50. When the operator operates the start icon 52 through the mouse 11a, countdown of the timer icon 51 is started. Note that the operator at this time has already captured the X-ray image, and the X-ray image for diagnosis is displayed in the image display area 50. This X-ray image is one of two images captured. Therefore, the DIP imaging is subsequently performed. When the time measurement by the timer icon 51 ends and step S20 ends, the process returns to step S15.

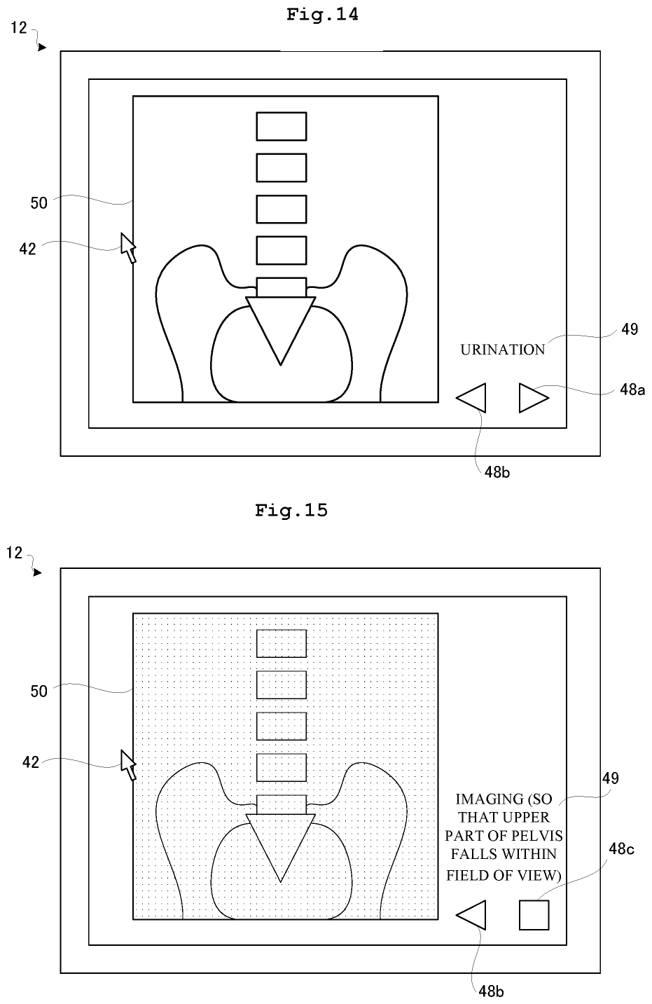

The processing returns to step S15 when step S20 ends, but at this time, the operator is in the middle of imaging and does not instruct to end the procedure display, and thus the process proceeds to step S16. Then, steps S15 and S16 are repeated until the operator operates the forward icon 48a or the backward icon 48b through the mouse 11a. When the operator operates the forward icon 48a through the mouse 11a, the process proceeds to step S17. At this time, since the timer icon is displayed on the display unit 12, the timer icon 51 and the start icon 52 are deleted from the display unit 12 in this step. Then, the process proceeds to step S18, and the display unit 12 displays “urination”, which is the fourth item in the description list L1, as a comment 49 at the end of the screen as illustrated in FIG. 14. In this manner, the character string described by the operator for Procedure 4 is displayed on the display unit 12, and the displayed procedure is updated. That is, the display of the comment 49 is updated from Procedure 3 to Procedure 4.

Thereafter, the process proceeds to step S19, but since the numerical value corresponding to the imaging type 1 in the timer setting value association table T2 is 3 and the fourth procedure is displayed in FIG. 14, the determination is false and the process returns to step S15. However, at this time, the operator is in the middle of imaging and does not instruct to end the procedure display, and thus the process proceeds to step S16. Then, steps S15 and S16 are repeated until the operator operates the forward icon 48a or the backward icon 48b through the mouse 11a. When the operator operates the forward icon 48a through the mouse 11a, the process proceeds to step S17. At this point of time, since the timer icon is not displayed on the display unit 12, nothing happens. Then, the process proceeds to step S18, and as illustrated in FIG. 15, the display unit 12 displays “imaging (so that the upper part of the pelvis fits in the field of view)”, which is the fifth item of the description list L1, as a comment 49 on the end of the screen. In this manner, the character string described by the operator for Procedure 5 is displayed on the display unit 12, and the displayed procedure is updated. That is, the display of the comment 49 is updated from Procedure 4 to Procedure 5.

The operator at this time of FIG. 15 is in the middle of capturing a live image with the support of the comment 49 so that the upper part of the pelvis falls within the field of view, and a fluoroscopic image (live image) of the subject is displayed in the image display area 50. At this time, around the comment 49 on the display unit 12, instead of the forward icon 48a for sending the item, an end icon 48c for ending the procedure display is displayed while avoiding the image display area 50. The end icon 48c can be operated by the operator through the mouse 11a, similarly to the forward icon 48a and the backward icon 48b.

Thereafter, the process proceeds to step S19, but since the numerical value corresponding to the imaging type 1 in the timer setting value association table T2 is 3 and the fifth procedure is displayed in FIG. 15, the determination is false and the process returns to step S15. When the operator operates the end icon 48c through the mouse 11a at this time point, the determination in step S15 is true, and the process proceeds to step S21.

Step S21: The comment 49, the backward icon 48b, and the end icon 48c are deleted from the display unit 12, and the process ends. Thereafter, the operator captures the second X-ray image, and the imaging itself by the imaging type 1 is also ended.

As described above, according to the X-ray imaging apparatus 10 of the embodiment, the support for the operator can also be completed by the apparatus configuration. That is, according to the embodiment, it is possible to flexibly support the operator for each imaging type. This is because the operator can appropriately set the reference content according to his/her convenience through the console 11. The apparatus according to the embodiment assists an operator by acquiring an imaging type to be executed and performing display matching the imaging type. Therefore, it can be said that the X-ray imaging apparatus according to the present invention is optimal in terms of hygiene without requiring the operator to rely on a tool outside the apparatus. In addition, the configuration of the embodiment has high flexibility according to the convenience of the operator. This is because a different imaging type can be created and stored for each operator. Since the description list is different for each imaging type, the operator can prepare a dedicated description list. Therefore, the X-ray imaging apparatus can perform display according to the operator according to the description list on the display unit 12.

### Effects of Configuration of Embodiment

Hereinafter, a configuration and an effect of the X-ray imaging apparatus 10 in this example will be described.

(1) The X-ray imaging apparatus 10 of the present example includes a console 11 that causes an operator's description to be input, an information processing unit 13 that associates an imaging type with an operator's description content, a memory 21 that stores the operator's description content in association with the imaging type, an imaging type acquisition unit 14 that acquires an imaging type to be executed, and a display unit 12 that displays the operator's description content corresponding to the acquired imaging type.

According to the configuration according to (1) described above, the description of the operator is input, and the description is associated with the imaging type. Since the operator can freely describe, the information related to the imaging type can be held in the memory 21 according to the convenience of the operator. Then, in a case where imaging for a certain imaging type is executed, the display unit 12 displays the description content of the operator corresponding to the imaging type to be executed. Since the operator can perform imaging while referring to the display content, smooth and reliable imaging can be performed with appropriate support.

Moreover, since the support regarding the photographing does not depend on a tool outside the apparatus, the imaging is optimized in terms of hygiene.

(2) The above-described display unit 12 updates the items in the order of the description list on the basis of the description list including a plurality of items as the description content of the operator and the forward feed instruction by the operator, and updates the items by tracing back the description list on the basis of the description list and the reverse feed instruction by the operator.

According to the above-described configuration, it is possible to easily re-execute the procedure by reversely sending the comment 49 on the display unit 12. In addition, it is also possible to refer to the procedure related to the execution again by sending the comment 49 forward and confirming the procedure before actually executing the procedure and then sending the comment 49 backward.

(3) The above-described description of the operator is a character string indicating the imaging procedure.

According to the above-described configuration, the operator can reliably execute imaging while confirming the imaging procedure. It is easier to understand the description of the imaging procedure by characters. In addition, the data size is smaller than that of a figure such as a picture. Therefore, according to the above-described configuration, the operator can reliably and quickly understand the imaging procedure and start imaging.

(4) The above description of the operator is a character string indicating the check content of the operator in imaging.

According to the above-described configuration, the operator can reliably execute imaging while confirming the check content. It is easier to understand the check content referred to by the operator by describing the check content using characters similarly to the above-described imaging procedure. Therefore, according to the above-described configuration, the operator can reliably and quickly understand the check content at the time of imaging and start imaging.

(5) The above-described display unit 12 displays the timer icon 51 capable of measuring time when displaying the description content of the operator.

According to the above-described configuration, since the time measurement can be completed by the X-ray imaging apparatus 10, the operator does not need to separately prepare a watch for the time measurement. Since the support regarding the photographing does not depend on a tool outside the apparatus, the imaging is optimized in terms of hygiene.

(6) The above-described display unit 12 can display a captured image.

According to the above-described configuration, information for supporting image capturing is displayed on the display unit 12 on which the captured image is displayed. Therefore, the operator can perform imaging while referring to the content described in the comment 49 without greatly moving the line of sight.

(7) The X-ray imaging apparatus 10 of the present example includes a console 11 that causes an operator to input a setting value of time, an information processing unit 13 that associates an imaging type with the setting value, a memory 21 that stores the setting value in association with the imaging type, an imaging type acquisition unit 14 that acquires an imaging type to be executed, and a display unit 12 that displays a timer icon 51 capable of measuring time indicated by the setting value corresponding to the acquired imaging type.

According to the above-described configuration, the setting value of the time by the operator is input, and the setting value is associated with the imaging type.

Since the operator can freely input the setting value, the timer measurement time according to the imaging type can be held in the memory 21 according to the convenience of the operator. Then, in a case of executing imaging for a certain imaging type, the display unit 12 displays a timer icon 51 capable of measuring time indicated by a setting value corresponding to the imaging type to be executed. Since the operator can perform imaging while referring to the timer icon 51, smooth and reliable imaging can be performed with appropriate support. Moreover, since the support regarding the photographing does not depend on a tool outside the apparatus, the imaging is optimized in terms of hygiene.

(8) The above-described timer icon 51 displays a countdown based on the setting value.

According to the above-described configuration, since the operator can wait until the lapse of the predetermined time while referring to the remaining time, the operator can reliably know the lapse of time.

(9) The timer control unit 15 that starts time measurement of timer icon 51 on the basis of the above-described instruction to start measurement by the operator is provided.

According to the above-described configuration, since the timer icon 51 starts the operation according to the instruction of the operator, the operator can start the measurement of time in accordance with the progress of the work.

### Other Embodiments

Note that the embodiments disclosed herein are illustrative in all respects and are not restrictive. The scope of the present invention includes the claims and all modifications within the meaning and scope equivalent to the claims. As an example, the present invention can be modified as follows.

(1) The configuration of the embodiment includes both the description list and the timer setting value association table T2, but the present invention is not limited to this configuration. The present invention can also be applied to a configuration in which the comment 49 corresponding to the inspection type is displayed on the display unit 12 and the timer icon 51 is not displayed. In this case, the timer setting value association table T2 is not necessarily required. In addition, it is not necessary to display the character string 45a, the numerical value input box 45b, the character string 45c, the setting value input box 45d, and the character string 45e on the list creation screen in the pre-imaging setting processing described with reference to FIGS. 7 and 8.

(2) The configuration of the embodiment includes both the description list and the timer setting value association table T2, but the present invention is not limited to this configuration. The present invention can also be applied to a configuration in which the timer icon 51 is displayed on the display unit 12 at the start of imaging and the comment 49 is not displayed. In this case, the column of the corresponding item in the description list and the timer setting value association table T2 is not necessarily required. In addition, the forward icon 48a, the backward icon 48b, and the end icon 48c may not be displayed on the display unit 12. It is not necessary to display the column 43 and the input box 44 on the list creation screen in the pre-imaging setting processing described with reference to FIGS. 7 and 8. In the present example, it is also possible to adopt a configuration in which the timer icon is deleted from the screen of the display unit 12 when the measurement of time ends.

(3) In the configuration of the embodiment, the items of the description list are updated by the operations of the forward icon 48a and the backward icon 48b, but all of the description list may be displayed on the display unit 12 in step S14 in the description content display processing. In this case, steps S16 to S19 in the description content display processing are not necessarily required. It is not necessary to display the plurality of input boxes 44 on the list creation screen in the pre-imaging setting processing described with reference to FIGS. 7 and 8.

(4) In the configuration of the embodiment, the character string input by the operator through the keyboard 11b is displayed on the display unit 12 as the comment 49, but the comment 49 may be a figure described by the operator through the mouse 11a.

(5) Although the display unit 12 of the embodiment can display the captured image, another monitor that displays the captured image can be provided in addition to the display unit 12.

(6) The timer icon of the embodiment notifies the passage of time to the operator by counting down, but the timer icon can be replaced with a configuration of counting up.

(7) The start icon 52 for starting the timer is displayed around the timer icon of the embodiment, but the timer icon may not include the start icon 52, and the timer icon may automatically start measuring the time.

### REFERENCE SIGNS LIST

1 top plate
2 strut
3 X-ray tube
4 detector
5 collimator
11 console (description input unit, setting value input unit)
12 display unit
13 information processing unit (description content processing unit, setting value processing unit)
14 imaging type acquisition unit
15 timer control unit
16 image generation unit
17 strut movement control unit
18 strut moving mechanism
19 X-ray tube control unit
21 memory
51 timer icon (timer)

1. A medical imaging apparatus comprising:
a description input unit that inputs a description of an operator;
a memory that stores the description content of the operator in association with the an imaging type;
an imaging type acquisition unit that acquires an imaging type to be executed; and
a display unit that reads out from the memory and displays the description content of the operator corresponding to the acquired imaging type.

2. The medical imaging apparatus according to claim 1, wherein the display unit updates items in an order of a list based on a list including a plurality of items which are description contents of the operator and a forward feed instruction by the operator, and updates the items by tracing back the list based on the list and a reverse feed instruction by the operator.

3. The medical imaging apparatus according to claim 1, wherein the description of the operator is a character string indicating an imaging procedure.

4. The medical imaging apparatus according to claim 1, wherein the description of the operator is a character string indicating a check content of the operator in imaging.

5. The medical imaging apparatus according to claim 1, wherein the display unit displays a timer capable of measuring time when displaying the description content of the operator.

6. The medical imaging apparatus according to claim 1, wherein the display unit is capable of displaying a captured image.

7. A medical imaging apparatus comprising:
a setting value input unit that inputs a setting value of time by an operator;
a memory that stores the setting value in association with the imaging type;
an imaging type acquisition unit that acquires an imaging type to be executed; and
a display unit that displays a timer capable of measuring time indicated by a setting value corresponding to the acquired imaging type, the setting value being read out from the memory.

8. The medical imaging apparatus according to claim 7, wherein the timer performs a countdown display based on the setting value.

9. The medical imaging apparatus according to claim 7, comprising a timer control unit that starts time measurement of a timer based on an instruction to start measurement by the operator.
